(19) United States
(12) Patent Application Publication
(10) Pub. No.: US 2004/0259124 A1
(43) Pub. Date: Dec. 23, 2004

(54) Methods for oligonucleotide probe design

(75) Inventors: Stefan Bekiranov, Oakland, CA (US); Rui Mei, Santa Clara, CA (US); Mei-Mei Shen, Cupertino, CA (US); Teresa A. Webster, Santa Clara, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/782,614

(22) Filed: Feb. 19, 2004

Related U.S. Application Data
(60) Provisional application No. 60/448,741, filed on Feb. 19, 2003.
(60) Provisional application No. 60/458,141, filed on Mar. 26, 2003.
(60) Provisional application No. 60/448,781, filed on Feb. 20, 2003.

(51) Int. Cl. C12Q 1/68; G06F 19/00; G01N 33/48; G01N 33/50

(52) U.S. Cl. 435/6; 702/20

(57) ABSTRACT

The present invention includes methods for predicting the quantitative response of probes on a microarray. In a preferred embodiment, sequence dependent parameters are predicted and then used to predict probe response.

### REFERENCES TO RELATED APPLICATIONS

The present application claims priority to U.S. Provisional Application Ser. Nos. 60/448,741 filed on Feb. 19, 2003 and 60/458,141 filed on Mar. 26, 2003 and is related to U.S. patent application Ser. Nos. 09/718,295, 10/017,034 (currently abandoned), Ser. Nos. 10/308,379, 10/310,013 and U.S. Provisional Application Ser. Nos. 60/335,012 and 60/493,185. All cited applications are incorporated herein by reference for all purposes.

### BACKGROUND OF THE INVENTION

Probes that exhibit a sensitive and predictable response to concentrations of their specific targets are desirable for quantitative detection of transcripts on microarrays. This response often occurs in the presence of a complex mixture of nonspecific targets. A good metric to ensure reproducible array performance is to select probes that are responsive to specific target and that are independent (i.e. the sequences of the different probes are preferably non-overlapping).

### SUMMARY OF THE INVENTION

In one aspect of the invention, methods, computer software and computer systems for selecting oligonucleotide probes are provided. The probes selected using the methods, software and systems are particularly suitable for being used as immobilized probes on a solid support, such as microarrays.

In preferred embodiments, the method uses the Langmuir adsorption isotherm model to relate intensity to target levels. In preferred embodiments, the Langmuir isotherm is used to related intensity to target concentration in experimental data. Sequence dependent parameters (such as ΔG*), Ln(Iₛₐₜ) are extracted from the experimental data. As used herein, Iₛₐₜ refers to the maximal intensity when all sites are occupied. The relationship between the sequence dependent parameters and probe sequence is used to predict sequence dependent parameters according to a candidate probe sequence.

The predicted parameters are related to probe responses. The candidate probes are then selected according to their predicted response. Computer software products and computer systems are also provided for performing the methods.

ΔG* is a linear transformation of ΔG_{d}, the desorption activation free energy. One aspect of the present invention provides a model for the sequence dependence of ΔG_{d}, which takes into account the positional contributions of each base and also the position contributions of runs of 5 C bases and runs of 4 G bases. Other models that capture the sequence contributions to ΔG_{d} may also be used for this step. Experimental data can be used to empirically establish models for predicting ΔG* and ΔG_{d}.

A metric for probe response can be defined to be the slope of the line, Ln-LnSlope, that relates Ln(I) to Ln([T]), where I is the hybridization intensity of a probe to its target in the presence of a complex genomic background. An empirical relationship between the ΔG* predicted and the Ln-LnSlope can be established (FIG. 2) and can be used to predict the Ln-LnSlope.

### BRIEF DESCRIPTION OF THE DRAWINGS

The accompanying drawings, which are incorporated in, and form a part of this specification, illustrate embodiments of the invention and, together with the description, serve to explain the embodiments of the invention.

FIG. 1A shows the Langmuir-like behavior of I vs [T] for several probes.

FIG. 1B shows a simulated Langmuir curve.

FIG. 2 shows the empirical relationship between the ΔG* predicted by the MLR model of data taken from spikes in a simple background and the Ln-LnSlope observed for the probes taken from data in a complex background.

FIG. 3 shows predicted and observed Ln-LnSlopes for the probes covering two YTC genes. The example in FIG. 3a has a correlation coefficient, 0.8, and average residual, 0.05; the example in FIG. 3b has correlation coefficient, 0.84, and average residual, −0.01.

FIG. 4 shows the relationship between average residual and observed Ln-Ln Slope.

### DETAILED DESCRIPTION OF THE PREFERRED EMBODIMENTS

In one aspect of the invention, methods, computer software and computer systems for selecting oligonucleotide probes are provided. The probes selected using the methods, software and systems are particularly suitable for being used as immobilized probes on a solid support, such as microarrays. In preferred embodiments, the method uses the Langmuir adsorption isotherm model to relate intensity to target levels. In preferred embodiments, the Langmuir isotherm is used to relate intensity to target concentration in experimental data. Sequence dependent parameters (such as ΔG*, Ln(Iₛₐₜ) are extracted from the experimental data. Sequence dependent parameters are predicted. The predicted parameters are related to probe responses. The candidate probes are then selected according to their predicted response. Computer software products and computer systems are also provided for the performing the methods.

I. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841 (currently abandoned), WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the company's website.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Iinis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and 6,582,938, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592, 6,632,611 and U.S. patent application Ser. Nos. 09/916,135, 09/920,491 and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Provisional Application Ser. No. 60/364,731 (presently abandoned) and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Provisional Application Ser. No. 60/364,731 (presently abandoned) and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable media include the floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

The present invention may also make use of the several embodiments of the array or arrays and the processing described in U.S. Pat. Nos. 5,545,531 and 5,874,219. These patents are incorporated herein by reference in their entireties for all purposes.

Additionally, the present invention may have preferred embodiments that include methods for providing genetic information over networks such as the Internet as shown in U.S. patent application Ser. No. 10/063,559 and U.S. Provisional Application Ser. Nos. 60/349,546 (presently abandoned), 60/376,003 (presently abandoned), 60/394,574, 60/403,381.

Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Array Plate or a Plate is a body having a plurality of arrays in which each array is separated from the other arrays by a physical barrier resistant to the passage of liquids and forming an area or space, referred to as a well.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs) as described in U.S. Pat. No. 6,156,501 that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a biopolymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Excitation energy refers to energy used to energize a detectable label for detection, for example illuminating a fluorescent label. Devices for this use include coherent light or non coherent light, such as lasers, UV light, light emitting diodes, an incandescent light source, or any other light or other electromagnetic source of energy having a wavelength in the excitation band of an excitable label, or capable of providing detectable transmitted, reflective, or diffused radiation.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about IM, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25° C., e.g., 750 mM NaCl, 50 mM NaPhosphate, 5 mM EDTA, pH 7.4 (5×SSPE)and a temperature of from about 25° C. to about 30° C.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning: A Laboratory Manual” 2^{nd} Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Label for example, a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Microtiter plates are arrays of discrete wells that come in standard formats (96, 384 and 1536 wells) which are used for examination of the physical, chemical or biological characteristics of a quantity of samples in parallel.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 20, 25, 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Reader or plate reader is a device which is used to identify hybridization events on an array, such as the hybridization between a nucleic acid probe on the array and a fluorescently labeled target. Readers are known in the art and are commercially available through Affymetrix, Santa Clara Calif. and other companies. Generally, they involve the use of an excitation energy (such as a laser) to illuminate a fluorescently labeled target nucleic acid that has hybridized to the probe. Then, the reemitted radiation (at a different wavelength than the excitation energy) is detected using devices such as a CCD, PMT, photodiode, or similar devices to register the collected emissions. See U.S. Pat. No. 6,225,625.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Reference will now be made in detail to exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

II. Methods for Oligonucleotide Probe Design

Probe selection and array design are important for the reliability, sensitivity, specificity, and versatility of microarrays. Basic probe selection methods, computer software and systems for expression microarrays are well-known in the art (Lockhart, et al. 1996. Expression Monitoring by hybridization to high-density oligonucleotide arrays. Nat. Biotechnol. 14: 1675-1680; U.S. patent application Ser. Nos. 09/718,295, 10/017,034 (currently abandoned), Ser. Nos. 10/308,379; 10/310,013 and U.S. Provisional Application Ser. Nos. 60/335,012 and 60/493,185, all of which are incorporated by reference).

Probe sets may be selected to represent each transcript, based on response, independence (degree to which probe sequences are non-overlapping), and uniqueness (lack of similarity to sequences in the expressed genomic background) using an optimization program, exemplary methods for which are described in U.S. patent application Ser. No. 10/308,379, which is incorporated herein by reference.

In one aspect of the invention, methods, computer software and computer systems for selecting oligonucleotide probes are provided. The probes selected using the methods, software and systems are particularly suitable for being used as immobilized probes on solid support, such as microarrays. In preferred embodiments, the method uses the Langmuir adsorption isotherm model to relate intensity to target levels. In preferred embodiments, the Langmuir isotherm is used to relate intensity to target concentration in experimental data. Sequence dependent parameters (such as ΔG*), Ln(Iₛₐₜ) are extracted from the experimental data. Sequence dependent parameters are predicted. The predicted parameters are related to probe responses. The candidate probes are then selected according to their predicted response. Computer software products and computer systems are also provided for performing the methods.

The Langmuir adsorption isotherm model is described in, for example, Masel, R. I. 1996. Principles of Adsorption and Reaction on Solid Surfaces. John Wiley and Sons, New York. Langmuir-like behavior of microarray hybridization has been noted previously (Naef et al. 2003. Absolute mRNA concentrations from sequence-specific calibration of oligonucleotide arrays. Nucleic Acids Research, Vol. 31(7): 1962-1968, incorporated herein by reference).

In some embodiments of the invention, which use the Langmuir adsorption isotherm models, the requirement for the number of models and terms are much smaller than other models. For example, in a preferred embodiment, the method of the invention requires only two models and 16 MLR terms. In contrast, for some model-based approaches, probe response prediction may involve 24 Multiple Linear Regression (MLR) models each with 86 terms.

In one aspect of the invention, a first order kinetic model of hybridization in simple background is presented, where it is assumed that a single target species hybridizes to a given probe. In one embodiment, the phenomenological kinetic parameters kₐ (adsorption rate or “on-rate”) and k_{d} (desorption rate or “off-rate”) may be related to free energy barriers of duplex formation using the Van't Hoff-Arrhenius form for an activated process. In other embodiments, a positional hydrogen bond model and a positional nearest-neighbour model relate the sequences of the target and probe to their free energy of binding. In yet other embodiments, the methods of the present invention may be applied to probe selection as well as expression analysis.

The following models, equations and derivations form the bases of the methods of the present invention.

Langmuir Adsorption Isotherm

The Langmuir isotherm was developed by Irving Langmuir in the early 1900s to describe the dependence of the surface coverage of an adsorbed gas on the pressure of the gas above the surface at a fixed temperature. The Langmuir isotherm model assumes monolayer adsorption on a homogeneous surface. There are many other types of isotherms (for example, Temkin, Freundlich, etc.) which differ in one or more of the assumptions made in deriving the expression for the surface coverage; in particular, on how they treat the surface coverage dependence of the enthalpy of adsorption. While the Langmuir isotherm is one of the simplest, it still provides a useful insight into the pressure dependence of the extent of surface adsorption (Source: Department of Chemistry website, Queen Mary College, University of London).

Extraction of ΔG* from Microarray Data

Duplex formation in the microarray system occurs between a probe with one end tethered to a surface and a target in solution. The target (T) hybridizes to its complementary probe (P) to form a target-probe duplex (T·P). The Langmuir adsorption isotherm for equilibrium conditions may be employed with the assumptions below, to obtain

θ=(kₐ[T])/(kₐ[T]+k_{d}) [1]

where

θ=[T·P]/[P] [2]

and

[T·P] is the surface concentration of target-probe duplexes. [P] is the total surface concentration of a feature, a set of probes with a common sequence covering a particular area on the array. [T] is the total concentration of intended target for a feature. Constants kₐ and k_{d} are rate constants for adsorption and desorption of the target to the probe feature, respectively. Equation 1 is based on the following assumptions. Adsorption occurs on specific features and all features are identical. The energy for adsorption is independent of how many of the surrounding features are occupied. Only one target occupies each feature and once all sites are occupied adsorption ceases. It is assumed that target-probe duplex formation/dissociation is an on-off process (i.e. nucleation and nucleotide zipper effects are ignored). Thus, the model predicts a two-state population of completely bound or unbound target molecules.

θ can be defined for the linear regime of Eq. 1, where [T]<<k_{d}/kₐ as

θ≅(kₐ/k_{d})[T] [3]

It has been found that kₐ has a relatively weak dependence on temperature and hence sequence of the probe from experiments on duplex formation of oligonucleotides in solution. One source of the modest sequence dependence is the nucleation barrier that should be sensitive to approximately five base pairs on the 5′ side of the probe (Bloomfield et. al. 2000. Nucleic Acids Structure, Properties, and Functions. Eds. Bloomfield, V. A., Crothers, D. M., Tinoco, I. University Science Books, Sausalito, Calif.).

In the present model, nucleation effects are ignored and it is assumed that kₐ does not depend on sequence. In contrast, the desorption rate, k_{d}, can vary by many orders of magnitude depending on the sequence of DNA and is very sensitive to temperature (Bloomfield et. al. 2000. Nucleic Acids Structure, Properties, and Functions. Eds. Bloomfield, V. A., Crothers, D. M., Tinoco, I. University Science Books, Sausalito, Calif.). This is expected theoretically from reaction rate theory (Hanggi et al. 1990. Reaction-rate theory: fifty years after Kramers. Reviews of Modern Physics 62: 251-341) where regardless of the dynamical regime that a system of reacting molecules is found (i.e. over-damped, under-damped), the desorption rate assumes a Van't Hoff-Arrhenius form

k^{d}=k_{d}⁰e^{-ΔG}^{_{d}}^{/RT*} [4]

where ΔG_{d} is the desorption activation free energy, T* is temperature, R is the molar gas constant, and k_{d}⁰ is a molecular relaxation rate which depends on the shape of the potential, viscosity of the medium etc.

Eq. (4) is both experimentally and theoretically well established for the case of simple molecules which react where ΔG_{d}/RT*>>1 (i.e. the condition of weak thermal noise). It has been shown that for short oligonucleotides in solution, this “on-off” model and the Arrhenius form give a reasonable description of the equilibrium population of bound and unbound molecules (Bloomfield et al. 2000. Nucleic Acids Structure, Properties, and Functions. Eds. Bloomfield, V. A., Crothers, D. M., Tinoco, I. University Science Books, Sausalito, Calif.).

Microarray data consists of fluorescent intensities (I) values, which are proportional to [T·P]

I=α[T·P]+b [5]

where b is background intensity not due to (T·P). The fraction of bound sites may be defined in terms of the observed intensity,

θ=(I−b)/(Iₛₐₜ−b) [6]

Combining Eqs. 3, 4, 6 gives

Ln(I−b)≅βΔG_{d}+ΔK+Ln(Iₛₐₜ−b)+Ln([T]) [7]

where ΔK=ln(kₐ/k_{d}⁰) and β=1/(RT*) are constants.

Eq. 7 may be rewritten as

Ln(I−b)=ΔG*+S* Ln([T]) [8]

where,

ΔG*=βΔG_{d}+ΔK+Ln(Iₛₐₜ−b)=the intercept of Ln(−b) vs. Ln([T]) [9]

and S*=the slope of Ln(I−b) vs. Ln([T]), which should be one for the linear regime.

A custom GeneChip® array, YTC, was designed that contained all 25 mer probe sequences to represent yeast transcripts (the targets of the array). The target transcripts were hybridized with the arrays at a range of concentrations according to a Latin square design. Hybridization data for this step was not generated in the presence of a genomic background (a mixture of labeled mRNA from human tissues). In some embodiments of the invention, the data was used to create a training set of approximately 50,000 ΔG* values by fitting the intercept values according to Eq. 9 for a set of approximately 50,000 probes covering 50 YTC transcripts. The range of [T] for the fit was 0.25 pM to 32 pM where a majority of the probes are found to be in the linear regime.

ΔG* in the linear regime was extracted by fitting the full Lagmuir form, Eq. 1, over the full target concentration range using Nonlinear Regression techniques, calculating k_{d}/kₐ and selecting concentrations that are reasonably below this concentration for fitting Eq. 8. In yet other embodiments, ΔG* values may be extracted by fitting the full Langmuir form over the full concentration range using Eqs. 1, 2, 4, 5 and 6. The value of b was estimated by extrapolating the response curve to zero concentration.

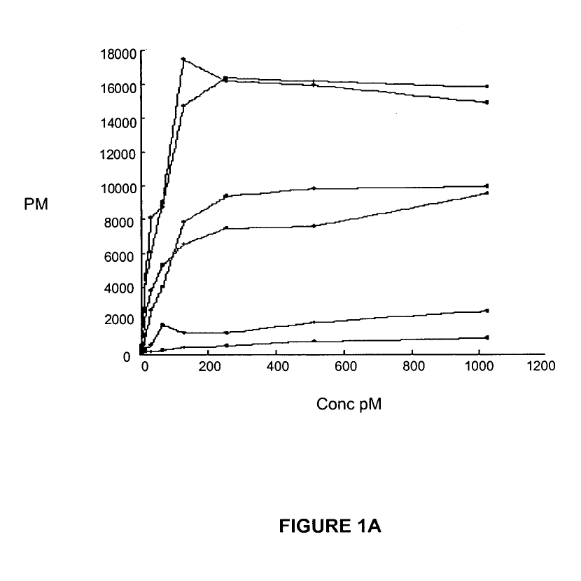

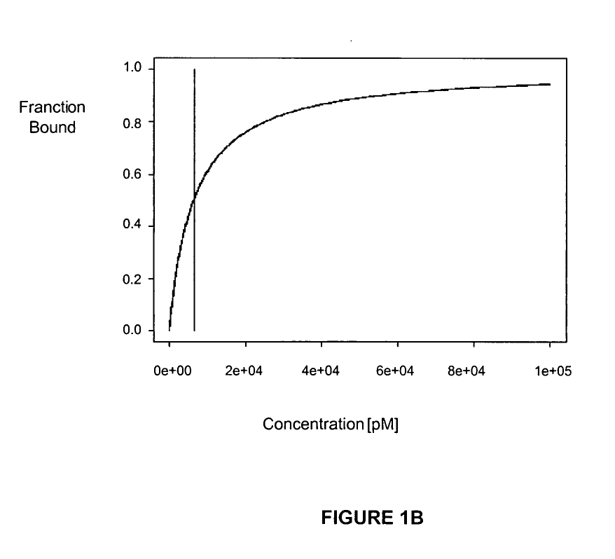

FIG. 1A shows the Langmuir-like behavior of I vs [T] for several probes. FIG. 1B shows a simulated Langmuir curve with the vertical bar indicating the boundary of the linear region. The bar in FIG. 1A shows the boundary of [T]=32 pM.

Prediction of ΔG* from Probe Sequence

ΔG* is a linear transformation of ΔG_{d} (Eq.9), the desorption activation free energy. ΔG_{d} is influenced by stacking energies and by hydrogen bonding between target-probe base pairs (Turner, 2000. Conformational Changes. In Nucleic Acids Structure, Properties, and Functions. Eds. Bloomfield, V. A., Crothers, D. M., Tinoco, I. pp. 259-334. University Science Books, Sausalito, Calif.). ΔG_{d} appears to depend not only on the compositions of the base pairs, but also on the positions of the probe bases relative to the ends of the probe. One aspect of the present invention provides a model for the sequence dependence of ΔG_{d}, which takes into account the positional contributions of each base and also the position contributions of runs of 5 C bases and runs of 4 G bases. Other models that capture the sequence contributions to ΔG_{d} may also be used for this step. A simple model consisting of nearest neighbor terms (Santa Lucia 1998. A unified view of polymer, dumbbell, and oligonucleotide DNA nearest-neighbor thermodynamics. Proc. Natl. Acad. Sci. USA 95: 1460-1465) captures some of the sequence components. A model consisting of the positional contributions of nearest neighbor terms is likely to be even more powerful.

In some embodiments, the contribution to ΔG* of bases and runs in each position can be expressed as a smooth function of probe base position, based on the positional relationship observed earlier.

$$Δ {G}^{*} = {W}_{1} {\sum }_{i = 1}^{N} {S}_{Ci} + {W}_{2} {\sum }_{i = 1}^{N} {S}_{Gi} + {W}_{3} + {\sum }_{i = 1}^{N} {S}_{Ti} + {W}_{1} {\sum }_{i = 1}^{N} {R}_{Ci} {S}_{Ci} + {W}_{5} {\sum }_{i = 1}^{N} {R}_{Gi} {S}_{Gi} + {W}_{6} {\sum }_{i = 1}^{N} {R}_{Ti} {S}_{Ti} + {W}_{7} {\sum }_{i = 1}^{N} {R}_{Ci}^{2} {S}_{Ci} + {W}_{8} {\sum }_{i = 1}^{N} {R}_{Gi}^{2} {S}_{Gi} + {W}_{9} {\sum }_{i = 1}^{N} {R}_{Ti}^{2} {S}_{Ti} + {W}_{10} {\sum }_{i = 1}^{N} {S}_{GGGG , i} + {W}_{11} {\sum }_{i = 1}^{N} {R}_{GGGG , i} {S}_{GGGG , i} + {W}_{12} {\sum }_{i = 1}^{N} {R}_{GGGG , i}^{2} {S}_{GGGG , i} + {W}_{13} {\sum }_{i = 1}^{N} {S}_{CCCCC , i} + {W}_{14} {\sum }_{i = 1}^{N} {R}_{CCCCC , i} {S}_{CCCCC , i} + {W}_{15} {\sum }_{i = 1}^{N} {R}_{CCCCC , i}^{2} {S}_{CCCCC , i} + {W}_{16} \qquad [ 10 ]$$

where N=Probe Length, i=Position {1, 2, . . . N} counting from the 3′ end of the probe sequence (or from left to right given the 11q sequence). X is a sequence of bases={C, G, T, GGGG, CCCCC}. Note X can be a single base.

$${S}_{xi} = \{ \begin{matrix}1 , if the lst Base of sequence , X , is in Position i \\ 0 , otherwise \end{matrix} \qquad [ 11 ]$$

Rᵢ=(i−MID)/(LEN−MID), [12]

SeqLen=number of bases in X. LEN=N−SeqLen+1. MID=Ceiling(LEN/2).

In one embodiment, using equation 10 as a model equation, the training set data consisting of ΔG*, and sequences for approximately 50,000 probes, multiple linear regression (MLR) was used to solve for the weights, Wⱼ, j=1-16 of Equation 10. This MLR solution to equation 10 can be used to predict ΔG* given a probe's sequence and the equations above. The correlation coefficient for ΔG*, predicted vs extracted ΔG* for the training set data was 0.8.

Prediction of Response Given ΔG*

A metric for probe response was defined to be the slope of the line, Ln-LnSlope, that relates Ln(I) to Ln([T]), where I is the hybridization intensity of a probe to its target in the presence of a complex genomic background. Latin Square data (in which YTC hybridized to target at known concentrations in a complex background) was used to obtain Ln(I) vs Ln([T]) profiles for the training set probes. These profiles were fitted for [T] ranging from 0.25 pM to 32 pM to obtain Ln-LnSlope values.

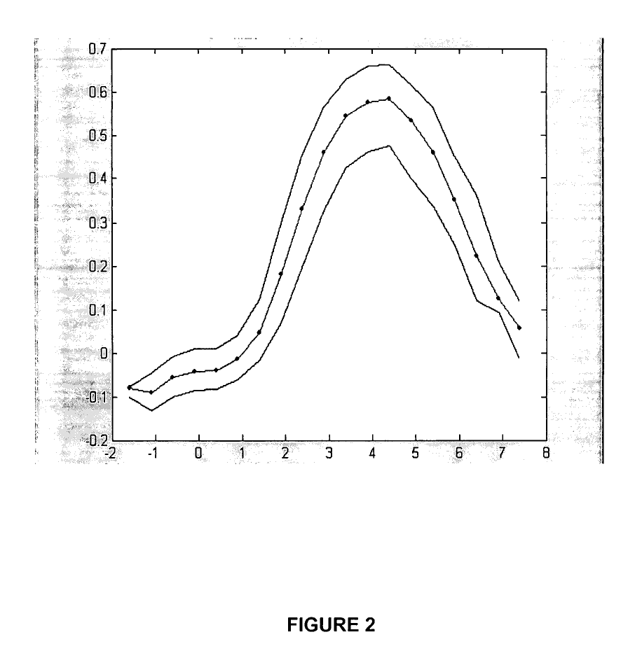

FIG. 2 shows that there is a well-defined empirical relationship between the ΔG* predicted by the MLR model of data taken from spikes in a simple background and the Ln-LnSlope observed for the probes taken from data in a complex background. It is observed that the free energy of hybridization or “affinity” of a probe is measurable in a simple background and cannot be directly measured in a complex background due to competing hybridization. One expects the low affinity probes to show poor slope response in both a simple and complex background because the temperature is too high for their target to remain bound. The high affinity probes are expected to show a poor slope response in complex background due to cross-hybridization of non-specific targets; however, their affinity can be measured in simple background and, as displayed in FIG. 2, probe slope response in a complex background may be predicted by using free energies derived from simple background. More specifically, each point in FIG. 2 is the median of points in a bin of predicted ΔG* of width=0.5. This relationship may be used to look up the Ln-LnSlope value, given a predicted ΔG, by interpolating between the nearest pairs of ΔG* points.

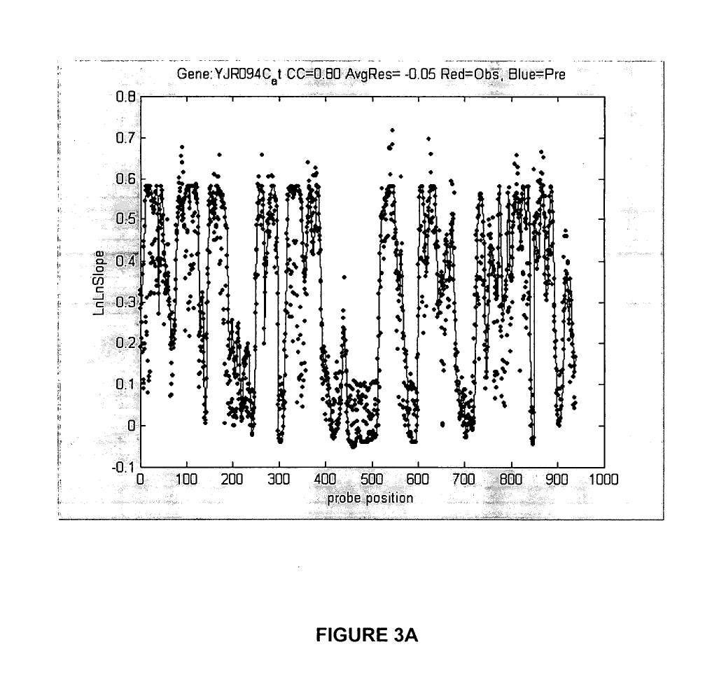

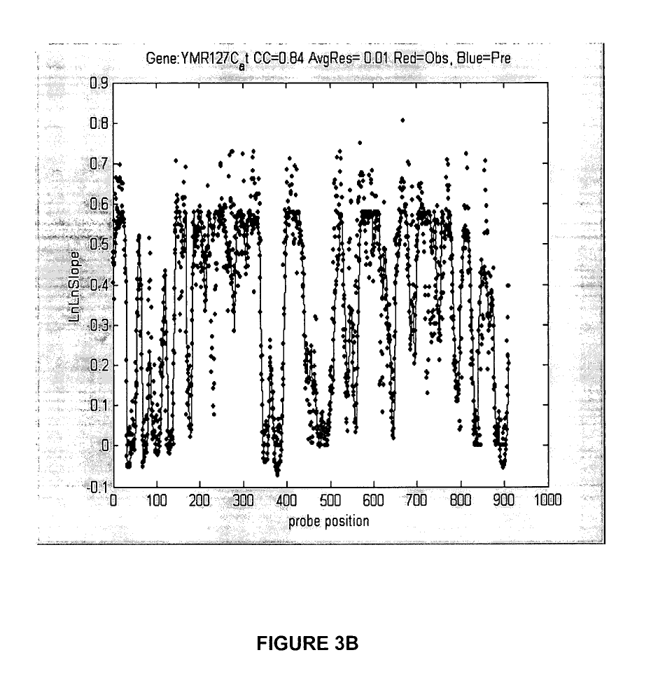

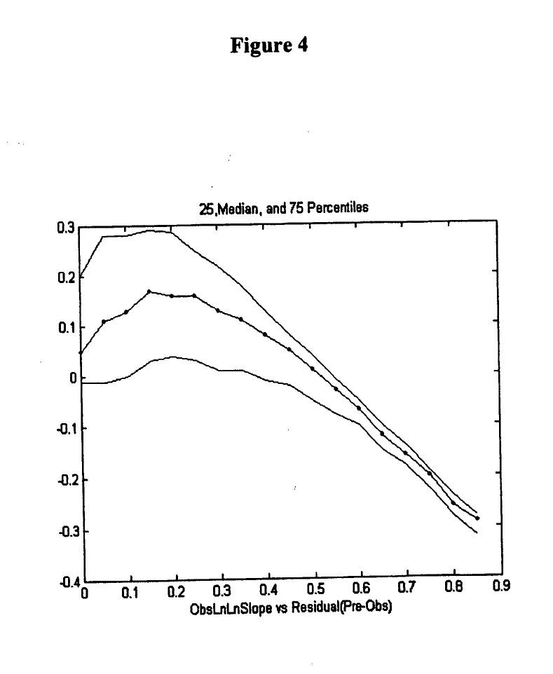

The training set data described above was used to build two models: (1) the 16 term MLR model, and (2) the predicted ΔG* vs. Ln-LnSlope profile. A test set of 49 YTC transcripts was created which was not used to build either model. The test set consisted of hybridization intensities for a given [T], collected on the arrays in the presence of complex background. For each probe in the test set, ΔG* was first predicted given the MLR model, and the Ln-LnSlope was then predicted using the ΔG* vs. Ln-LnSlope profile. The predictions were evaluated by comparing predicted vs observed Ln-LnSlopes for each set of probes covering each YTC transcript, and computing the correlation coefficient, and the average residual for Predicted vs. Observed Ln-LnSlopes. FIG. 3 shows an example of predicted and observed Ln-LnSlopes for the probes covering two YTC genes. The example in FIG. 3a has a correlation coefficient, 0.8, and average residual, 0.05; the example in FIG. 3b has correlation coefficient, 0.84, and average residual, −0.01. The average correlation coefficient for all 49 YTC genes is 0.74, and the average residual is −0.043. FIG. 4 shows the relationship between average residual and observed Ln-LnSlope. The residuals are lower when the Ln-LnSlope are low, which the critical range for predicting high quality probes. The approach tends to underpredict the high Ln-LnSlope.

It is to be understood that the above description is intended to be illustrative and not restrictive. Many variations of the invention will be apparent to those of skill in the art upon reviewing the above description. All cited references, including patent and non-patent literature, are incorporated herein by reference in their entireties for all purposes.

What is claimed is:

1. A computer implemented method for predicting probe response comprising
relating a sequence dependent parameter with probe response; and
predicting probe response for a value of the sequence dependent parameter.

2. The method of claim 1 wherein the sequence dependent parameter is ΔG*, wherein ΔG* is a free energy barrier.

3. The method of claim 2 wherein the probe response is the Ln(I)/Ln(T) slope, wherein, the I is the intensity in a complex background and T is target level.

4. The method of claim 3 wherein the relationship between probe response and ΔG* is established empirically.

5. The method of claim 4 wherein the ΔG* is predicted using a model relating ΔG* to probe sequence.

6. The method of claim 5 wherein the model is established by relating intensity to target levels using the Langmuir adsorption isotherm model in experimental data in simple background to extract sequence dependent parameters from the experimental data.
